(19) United States
(12) Patent Application Publication
(10) Pub. No.: US 2023/0248417 A1
(43) Pub. Date: Aug. 10, 2023

(54) ARTICULATING SURGICAL DEVICE

(71) Applicant: The Chinese University of Hong Kong, Shatin, N.T. (CN)

(72) Inventors: Kwok Wai Samuel AU, Shatin, N.T. (CN); Yan WANG, Fanling, N.T. (CN); Hoi Wut Yip, Shatin, N.T. (CN)

(21) Appl. No.: 18/010,192

(22) PCT Filed: Jun. 16, 2021
(86) PCT No.: PCT/CN2021/100271

§ 371 Date: Dec. 13, 2022

Related U.S. Application Data
(60) Provisional application No. 63/040,130, filed on Jun. 17, 2020.

(51) Int. Cl. A61B 18/14; A61B 34/30

(52) U.S. Cl. CPC A61B 18/1442; A61B 34/30; A61B 2017/00477

(57) ABSTRACT

A surgical device (600) includes an articulating portion (604) for navigating the device within a bodily cavity. The articulating portion (604) includes an articulating sheath (1000) and an articulating torque transmission wrist within the articulating sheath (1000). The articulating sheath (1000) may define one degree of freedom or two or more degrees of freedom. The articulating portion (604) may be part of a surgical device shaft (602) connected to an end effector (603) rotated by an internal torque transmission shaft including the articulating torque transmission wrist. The articulating portion (604) may be articulated while the torque transmission shaft is being rotated.

### FIELD OF INVENTION

The present technology relates to surgical devices for use in surgeries requiring access in confined anatomical regions, such as spinal surgery.

### BACKGROUND OF THE INVENTION

Surgical devices may include shafts with rotating ends used for drill and/or debridement. FIG. 1 shows a surgical drill 1 with a fixed straight shaft. Fixed straight shafts have the advantage of allowing the operator to apply force to the tool end due to the rigid fixed shaft. However, fixed straight shafts have the disadvantage of essentially requiring a direct straight line between the point of insertion and the target treatment location. Further, for drilling/debridement applications, the direction of boring is the same as the direction of insertion which is disadvantageous when a different drilling/debridement direction is desired.

In addition to fixed straight shafts, surgical drills may include a fixed bent shaft 2, as shown for example in FIG. 2. Similar to fixed straight shafts, fixed bent shafts have the advantage of allowing the operator to apply force to the tool end due to the rigid fixed shaft. Further, fixed bent shafts have the advantage of allowing the treatment site to be offset from the initial line of insertion. This may be beneficial for treatment sites that are behind an obstacle, for example a bone. However, due to the fixed angle of the bend, fixed bent shafts have the disadvantage of the bend angle being unchangeable, i.e. fixed, which may not be suitable for various anatomical variations, and further does not allow navigation through circuitous paths between a point of insertion and a treatment site.

Surgical devices 3, for example as shown in FIGS. 3 and 4, may include steering functionality that allows for navigation through circuitous paths. However, due to the flexible nature of the sheaths, these surgical devices 3 perform poorly in transmitting force to hold the tool end of the device against a target treatment site, for example pressing a drill bit into a bone.

FIG. 5 shows a shaft 5 comprising a sheath with a plurality of pinned joints and a flexible rotation shaft for drilling. The shaft 5 is limited by the flexible rotational shaft which is made of superelastic metal. As a result, the bending angle of one joint can only reach 30°, which limits the dexterity of the drill.

Accordingly, there is a need for a surgical device that can navigate through circuitous paths, including being able to bend greater than 30 degrees, and apply force to a target treatment site.

### SUMMARY OF THE INVENTION

A surgical device including an articulating portion for navigating the device within a bodily cavity. The articulating portion includes an articulating sheath and an articulating torque transmission wrist within the articulating sheath. The articulating sheath may define one degree of freedom or two or more degrees of freedom. The articulating portion may be part of a surgical device shaft connected to an end effector rotated by an internal torque transmission shaft including the articulating torque transmission wrist. The articulating portion may be articulated while the torque transmission shaft is being rotated.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 shows a surgical drill with a fixed straight shaft.

FIG. 2 shows a surgical drill with a fixed bent shaft.

FIGS. 3 and 4 show shafts including a flexible sheath and a flexible torque transmission shaft.

FIG. 5 shows a shaft comprising a sheath with a plurality of pinned joints and a flexible rotation shaft for drilling.

FIGS. 6 and 7 show an embodiment of a surgical device according to the present technology.

FIGS. 8A and 8B show an embodiment of a surgical procedure according to the

FIGS. 9A-H show an embodiment of a bi-centric geared articulating sheath according to the present technology.

FIGS. 10A-J show an embodiment of a bi-centric non-geared articulating sheath according to the present technology.

FIG. 11 shows an embodiment of a single axis articulating sheath according to the present technology.

FIGS. 12A-F show an embodiment of a 2 degree of freedom articulating sheath according to the present technology.

FIGS. 13A-C show an embodiment of a universal joint articulating wrist according to the present technology.

FIGS. 14A-F show an embodiment of a slotted ball joint articulating wrist according to the present technology.

FIGS. 15A-H show an embodiment of a saddle ball joint articulating wrist according to the present technology.

FIGS. 16A-E show an embodiment of a double hinge sliding joint articulating wrist according to the present technology.

FIGS. 17A-D show an embodiment of a beveled gear joint articulating wrist according to the present technology.

FIGS. 18A-B show an embodiment of an articulating portion comprising a bi-centric non-geared articulating sheath and a slotted ball joint articulating wrist according to the present technology.

FIGS. 19A-B show an embodiment of an articulating portion comprising a bi-centric non-geared articulating sheath and a saddle ball joint articulating wrist according to the present technology.

FIGS. 20A-B show an embodiment of two articulating portions each comprising a bi-centric non-geared articulating sheath and a slotted ball joint articulating wrist according to the

FIGS. 21A-D show an embodiment of an articulating portion comprising a 2 degree of freedom articulating sheath and a double hinge sliding joint articulating wrist according to the present technology.

FIGS. 22A-B show an embodiment of two articulating portions each comprising a 2 degree of freedom articulating sheath and a double hinge sliding joint articulating wrist according to the present technology.

FIGS. 23A-C show an embodiment of a drill bit collet according to the present technology.

FIGS. 24A-C and 25 show embodiments of user interfaces of an articulating portion of a surgical device shaft according to the present technology.

FIGS. 26A-K show embodiments of a surgical device with an anchoring system according to the present technology.

FIGS. 27A-E show an embodiment of a bi-centric articulating sheath according to the present technology.

FIGS. 28A-F show an embodiment of a 4 degree of freedom articulating wrist according to the present technology.

FIGS. 29A-C show an embodiment of an end effector an articulating portion of a shaft according to the present technology.

FIGS. 30A-D show an embodiment of a handheld surgical device according to the present technology.

### DETAILED DESCRIPTION OF THE INVENTION

Throughout this description for the purposes of explanation, numerous specific details are set forth in order to provide a thorough understanding of the many aspects and embodiments disclosed herein. It will be apparent, however, to one skilled in the art that the many aspects and embodiments may be practiced without some of these specific details. In other instances, known structures and devices are shown in diagram or schematic form to avoid obscuring the underlying principles of the described aspects and embodiments. Like reference numbers and designations in the various drawings indicate like elements.

The present technology relates to miniaturized steerable surgical instruments with inner high-speed rotational motion transmission mechanisms. The high-speed rotational motion transmission mechanisms may be combined with different end-effectors at the distal end of the device and used for different surgical applications, including but not limited to: drilling holes, debriding tissues, and turning screws. Further for example, the technology can be used in surgical bone work especially for those surgeries required to operate or access in confined anatomical regions, such as spinal surgery or ENT surgery.

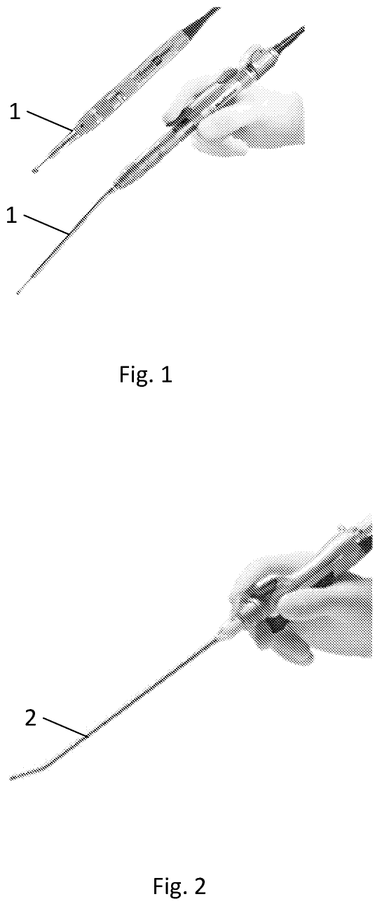

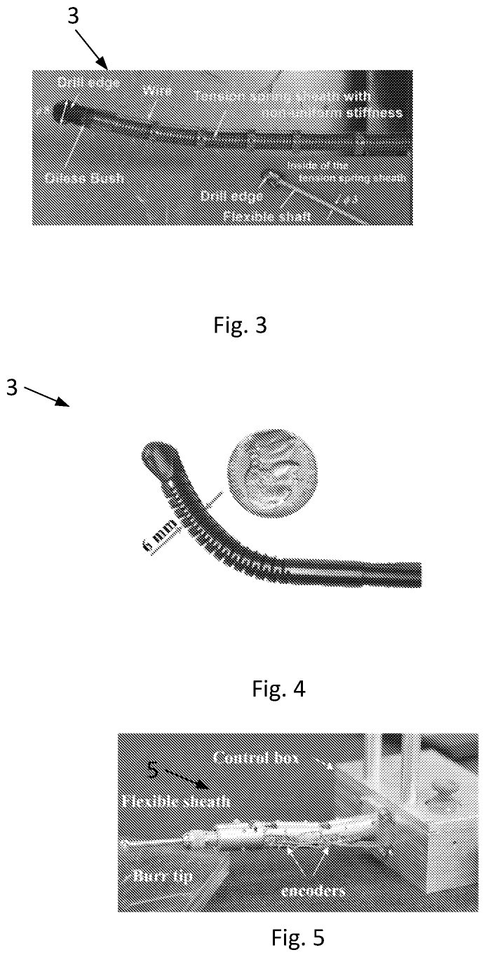

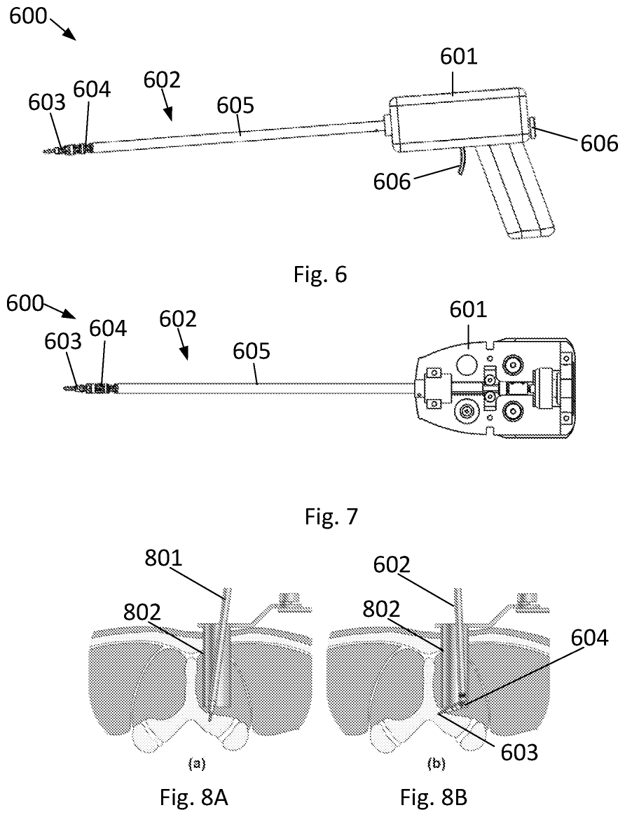

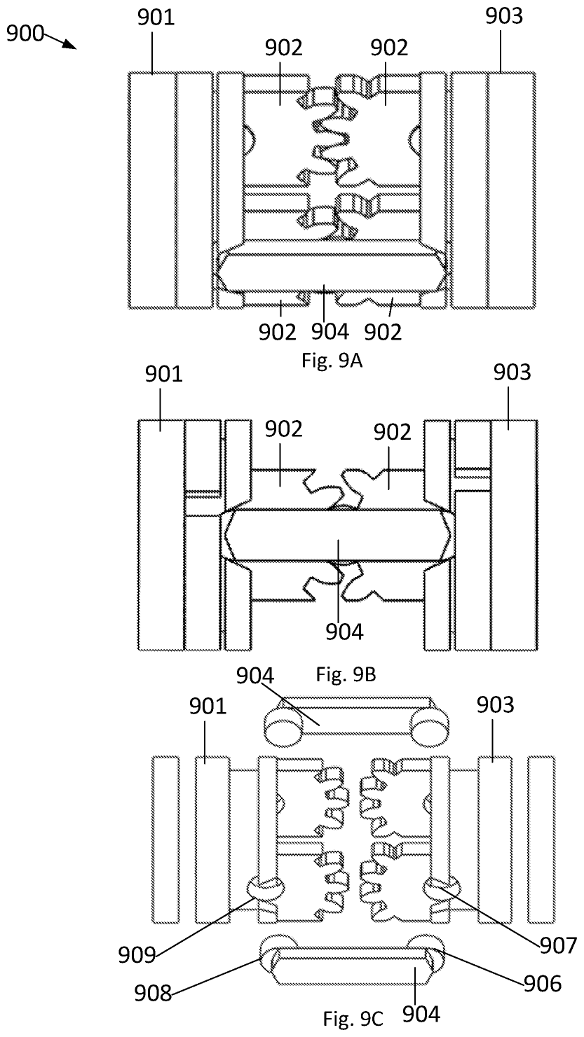

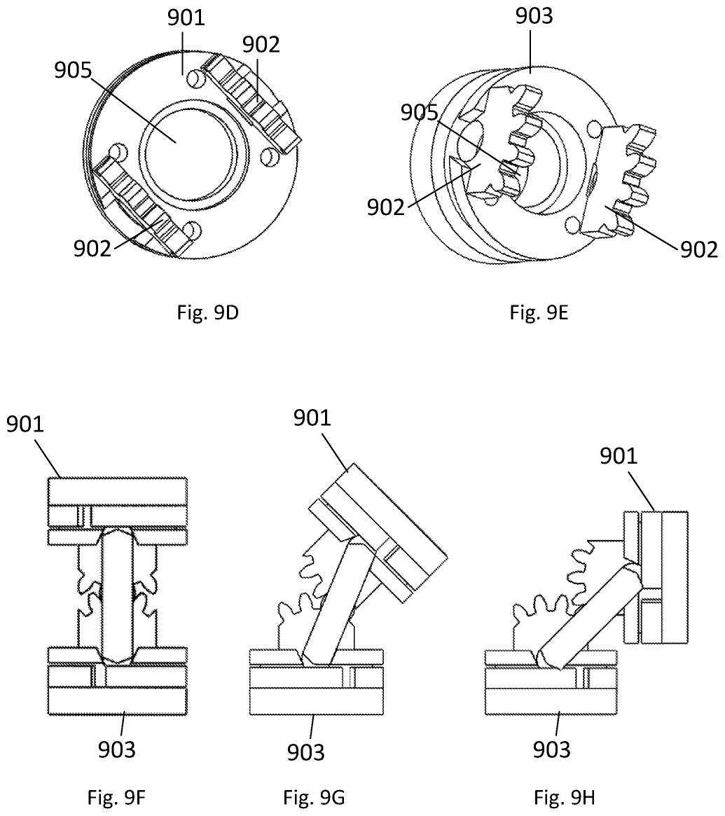

The disclosed technology provides the advantages of possessing higher dexterity and strength compared to the devices shown in FIGS. 1-5, offering tight degree bends with a small bending radius while having mechanical strength allowing for transmission of translation force even in a miniaturized scale or dimension. These advantages are beneficial in performing procedures in complicated surgical spots in that an articulating portion of a steerable shaft can be locked at an adjustable angle to provide stiffness and stabilization of the drill when dealing with hard bones. Further, in embodiments systems including articulating portions may further comprise adjustable anchoring systems with force sensing capability ensuring stable support for the distal end of the drilling wrist, enabling stable and precise drilling and debridement.

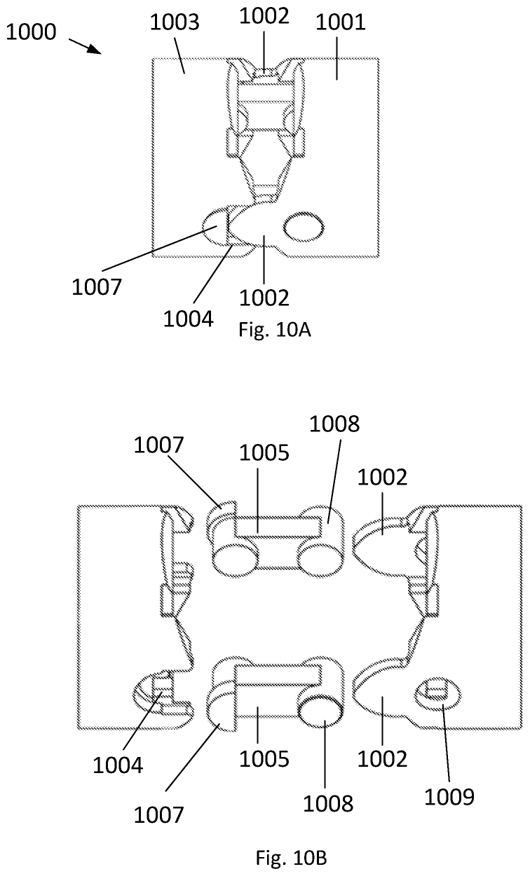

In embodiments, a surgical device may include a shaft with at least one articulating portion. For example as shown in FIG. 6, a surgical device 600 comprises a base portion 601, a shaft 602 and an end effector 603. The shaft 602 comprises a sheath portion encasing an inner torque transmission shaft, as will be discussed in more detail below. Motors in the base portion 601 may rotate the torque transmission shaft to cause the end effector 603 to rotate. The shaft 602 includes an articulating portion 604 and one or more rigid portions 605, each of which may include a sheath portion and an inner torque transmission shaft, as will be discussed in greater detail below. In embodiments, a shaft may have any number of articulating portions and rigid portions. The articulating portions allow for the end effector 603 to be moved relative to the base portion 601 in multiple degrees of freedom.

In embodiments, for example as shown in FIG. 6, the surgical device 600 may be a handheld device and the base portion 601 comprises a housing defining a handle for a user to hold. The housing of the base portion 601 may contain electronic circuitry and actuators for controlling manipulation of the articulating portions of the shaft assembly and for rotating the end effector. The base portion 601 may further comprise user input devices 606 connected to the electronics, for example knobs, triggers, buttons, thumb sticks. The user input devices 606 allow a user holding the device to control the articulation angle of the one or more articulating portions of the shaft, for example using a 2-DoF thumb stick interface. The base portion 601 may further comprise indicators for indicating device status to a user, for examples lights and/or screens. In embodiments, the base portion 601 may be part of an integrated robotic system.

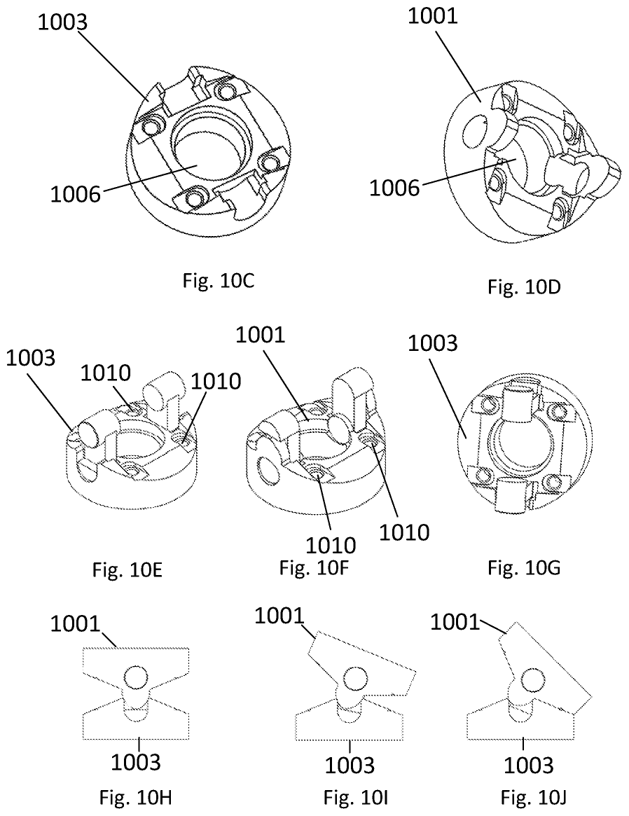

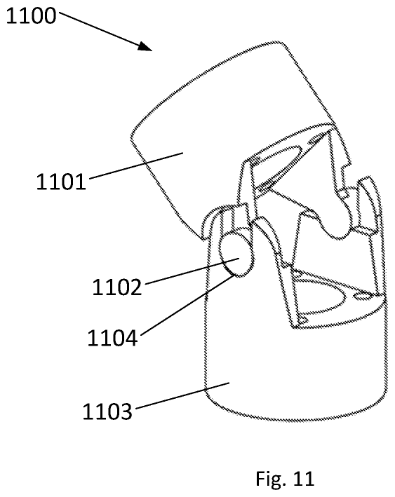

In embodiments, one or more articulating portions 604 may be located anywhere along the shaft 602 between the base portion 601 and the end effector 603. For example, as shown in FIGS. 6 and 7, one articulating portion 604 is located at a distal end of the shaft 602 and allows the shaft 602 to bend in order for the end effector 603 to be rotated to different angles to access a target treatment site that is not along the longitudinal axis of the rigid portion 605 of the shaft 602 between the articulating portion 604 and the base portion 601. For example, as shown in FIG. 8A, using a straight non-articulating shaft of a device 801, for example as shown in FIG. 1, in minimally invasive spinal surgery, the straight non-articulating shaft is inserted into a slender tubular retractor 802 and is limited in drilling position and drilling angle due to the rigid shaft contacting the tubular retractor 802. Using a shaft 602 with an articulating portion 604 according to the present technology, for example as shown in FIG. 8B, the drilling position and angle are less limited since the end effector 603, in this example a drill, can be steered, through one or more degrees of freedom via the articulating portion 604, to point in various directions relative to the rigid shaft portion constrained by the tubular retractor 802.

### Articulating Portion

In embodiments, an articulating portion may be coupled between one or more portions of rigid shaft and/or other articulating portions. Each articulating portion may allow for articulation in at least one degree of freedom, for example 1 degree of freedom, 2 degrees of freedom, or 3 or more degrees of freedom. The articulating portions may each include a plurality of bodies defining a number of degrees of freedom, wherein the degrees of freedom of an articulating portion may be defined as the sum of the degrees of freedom of the bodies comprising the articulating portion. For example, an articulating portion may each comprise two bodies each with two degrees of rotational freedom, therefore defining the articulating portion to have four degrees of freedom. Each articulating portion may comprise at least one articulating sheath, and at least one articulating wrist joint of a torque transmission shaft. The articulating sheaths may define an internal lumen housing the articulating wrist joints. The articulating wrist joints may be able to freely rotate within the articulating sheaths. In embodiments, an articulating wrist joint may be rotationally coupled to the articulating sheath, for examples with bearings in order to maintain the axial orientation of the articulating wrist relative to the articulating sheath while allowing for mutual articulation and relative rotations of the torque transmission shaft relative to the sheath.

### Articulating Sheaths

In embodiments, an articulating sheath of a sheath of a surgical device comprises a distal portion and a proximal portion coupled between other portions of the sheath and coupled together to allow for 1 or more degrees of freedom between the distal portion and the proximal portion, and therefore allow 1 or more degrees of freedom between other portions of the sheath. The distal and proximal portions may each be coupled to rigid portions of a shaft sheath and/or other articulating sheaths of adjacently coupled articulating portions. Each of the distal and proximal portions include an internal lumen. Further, elements for coupling the distal portions to the proximal portions maintain one or more continuous internal lumens through the articulating sheath in order to provide space for the torque transmission shaft, tendons, and wiring. Tendons or driving rods coupled to actuators, for example actuators in the base portion, may extend through one of the sheath lumens and be coupled to portions of the articulating sheath to control the articulation angle of the articulating sheath. In embodiments, the sheaths as disclosed herein may be used for microsurgery instruments and have a diameter of 5 mm or less.

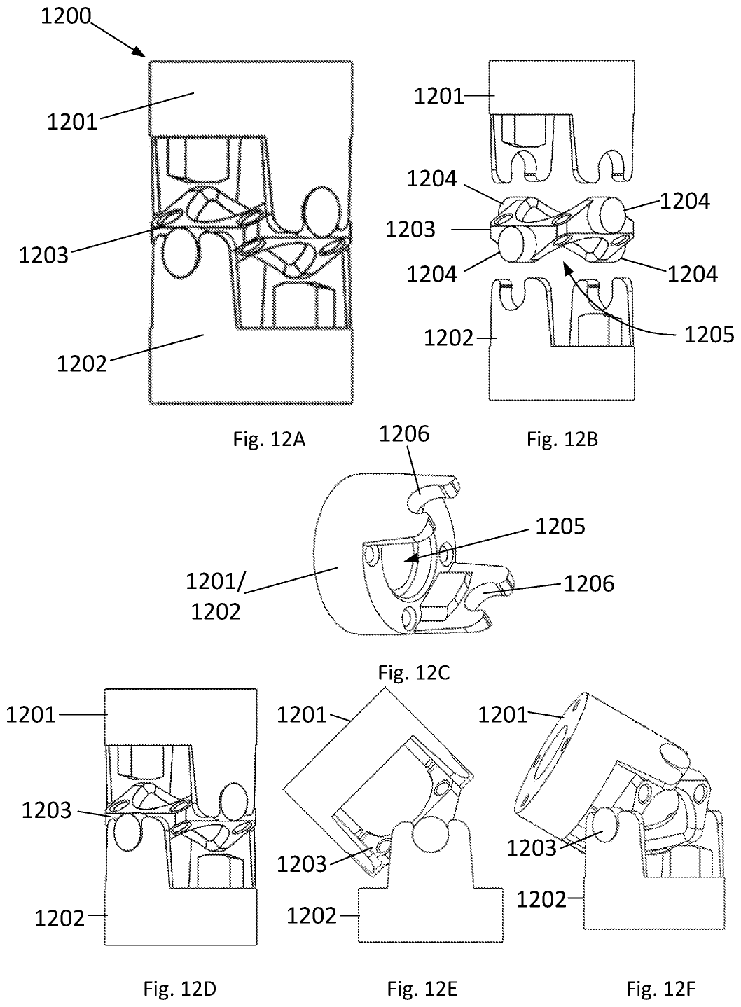

In embodiments, an articulating sheath may comprise a geared joint defining one degree of rotational freedom. In embodiments, the geared joint may be a bi-centric geared joint, for example as shown in FIGS. 9A-9H. Bi-centric joints comprise two axes of rotation with a rotation coupling interface to combine the two axes into a single degree of freedom. As shown in the exploded view in FIG. 9C the bi-centric geared joint 900 includes a distal portion 901 comprising two geared projections 902, and a proximal portion 903 comprising two geared projections 902. As shown in the assembled views of FIGS. 9A and 9B, the geared projections 902 mesh, and meshing is maintained through two dumbbell linkages 904. Each dumbbell linkage comprises a proximal axle 906 rotationally coupled to a bore 907 in a geared projection 902 of the proximal portion 903 and a distal axle 908 rotationally coupled to a bore 909 in a geared projection 902 of the distal portion 901. The axles 906 and 908 of the dumbbell may be held in place with respective bands positioned within grooves extending radially around the distal portion 901 and proximal portion 903 of the articulating sheath.

As shown in FIGS. 9D and 9E, the distal and proximal portions 901 and 903 each include a central through hole 905 between the geared projections 902 and extending in a longitudinal direction of the bi-centric geared joint 900. The central through holes 905 define a portion of one of the lumens of the articulating sheath. Further as shown, the dumbbells 904 may be coupled to the geared projections 902 outside of the central lumen defined in part by the central through holes 905.

FIGS. 9F-9H show a series of positions in order to show the relative movement between the distal portion 901 and proximal portion 903. As shown, the meshed gears and dumbbell connections allow for 1 degree of freedom articulation between the distal portion 901 and the proximal portion 903. The one degree of freedom allows for angles between 0 degrees as shown in FIG. 9F, 90 degrees as shown in FIG. 9H, and any angle in between, including 45 degrees as shown in FIG. 9G. The bi-centricity of the joint defines an increased radius of curvature of the internal lumen of the articulating sheath relative to a single axis joint. Further, a bi-centric geared joint provides high torsional and axial resistance to the external disturbance force and torque, while defining a large internal lumen to allow a torque transmission mechanism and tendons and rods to extend through the joint.

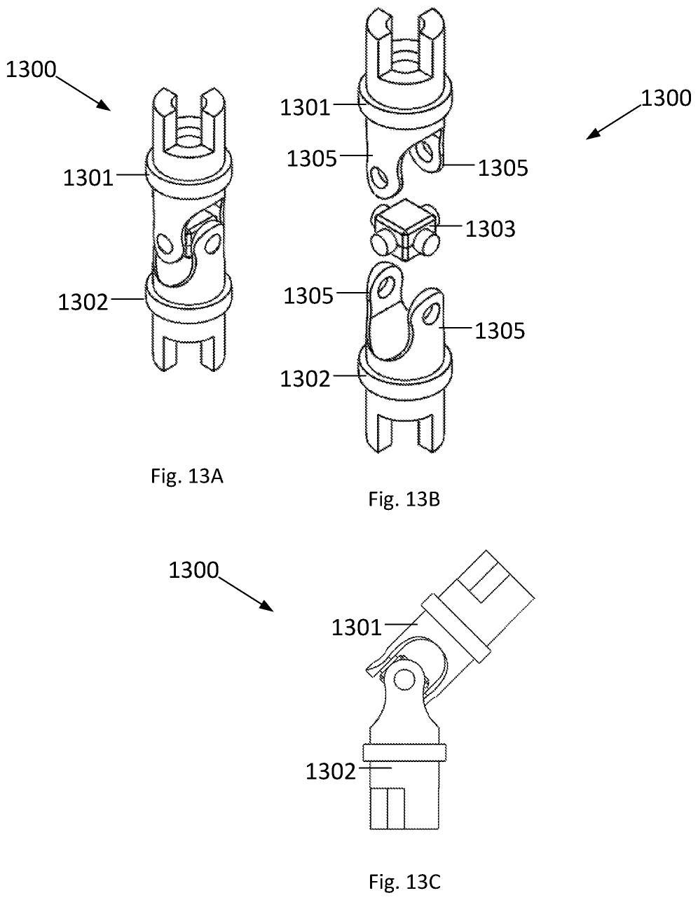

In embodiments, an articulating sheath may comprise a bi-centric non-geared joint, for example as shown in FIGS. 10A-10J. As shown in the exploded view of FIG. 10B the bi-centric joint 1000 includes a distal portion 1001 comprising two teeth 1002, and a proximal portion 1003 comprising two notches 1004. As shown in the assembled view of FIG. 10A, the teeth 1002 are complementary to and mesh within the notches 1004, and meshing is maintained through two dumbbell linkages 1005. Each dumbbell linkage comprises a proximal axle 1007, defined by a half disc, rotationally positioned within one of the notches. Each dumbbell linkage also comprises a distal axle 1008 rotationally coupled to a bore 1009 in the teeth 1002. In embodiments, orientations of the joint may be flipped so that the teeth are on the proximal portion and the notches on the distal portion. As shown in FIG. 10A, the proximal axles 1007 may include a flat portion so that the teeth 1002 do not contact the proximal axles.

As shown in FIGS. 10C-10E, the distal portion 1001 and proximal portion 1003 each include a central through hole 1006 between portions housing the axles of the dumbbells. The central through holes 1006 define a portion of the lumen of the articulating sheath. In embodiments, the distal portions and proximal portions, may include radial through holes for receiving rods and tendons. The radial through holes may be positioned around the central through hole. For example, as shown in FIGS. 10E and 10F, the distal portion 1001 and proximal portion 1003 each define corresponding radial through holes 1010 around the central through hole 1006.

As shown in FIGS. 10H-10J, the meshed teeth 1002, notches 1004 and dumbbell 1005 connections allow for 1 degree of freedom articulation between the distal and proximal portions. The one degree of freedom allows for angles between 0 degrees as shown in FIG. 10H, 45 degrees as shown in FIG. 10J, and any angle in between, including 22.5 degrees as shown in FIG. 10I. Similar to the joint of FIGS. 9A-9H, the bi-centricity of the joint of FIGS. 10A-10J defines an increased radius of curvature of the internal lumen of the articulating sheath relative to a single axis joint. The teeth 1002 and notches 1004 are advantageous in manufacturing joints with small dimensions. The teeth 1002 and notches 1004 of a bi-centric non-geared joint are further advantageous in being stronger relative to joints of similar dimensions including relatively small interfacing components. For example, relatively small interfacing components, for example multiple meshed gear teeth, may cause the joint to be able to resist lower external torques or forces due to the lower structural strength of each individual small interfacing component compared to the relatively large interfacing components of the bi-centric non-geared joint.

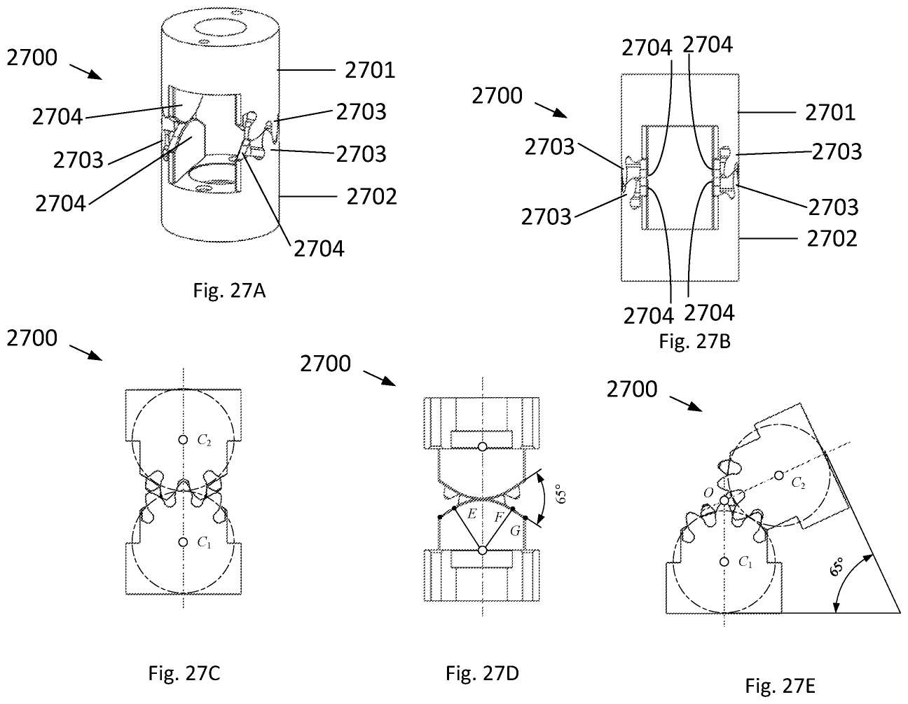

In embodiments, an articulating sheath may comprise a bi-centric joint with both geared and non-geared portions defining the bicentricity, for example as shown in FIGS. 27A-E. The bi-centric sheath 2700 includes a distal portion 2701 and a proximal portion 2702. The distal portion 2701 and the proximal portion 2702 each may have two geared portions 2703 and two curved surfaces 2704. The geared portions 2703 of the distal portion 2701 engages with the geared portion 2703 of the proximal portion 2702, as shown in FIG. 27C, and the curved surfaces 2704 of the distal portion engages with curved surfaces 2704 of the proximal portion 2702 and roll against each other, as shown in FIG. 27D, in order to define the bi-centricity of the sheath 2700. The sheath 2700 as shown only consist of two parts, which is advantageous for manufacturing purposes. The geared portions 2703 and curved surfaces 2704 may allow for rotation in each direction up to 80 degrees, for example 65 degrees in each direction. Engagement of the geared portions 2703 with each other prevents translational movement between the distal portion 2701 and the proximal portion 2702 in a first direction perpendicular to the longitudinal axis of the joint, and engagement of inner faces of the geared portions 2703 with other faces of the curved surfaces 2704 prevents translational movement between the distal portion 2701 and the proximal portion 2702 in a second direction perpendicular to the longitudinal axis of the joint and the first direction. Furthers, tendons and robs used to articulate the joint 2700, and other articulating portions in a system, may extend through the joint 2700 and also prevent the distal portion 2701 and the proximal portion 2702 from pulling apart axially.

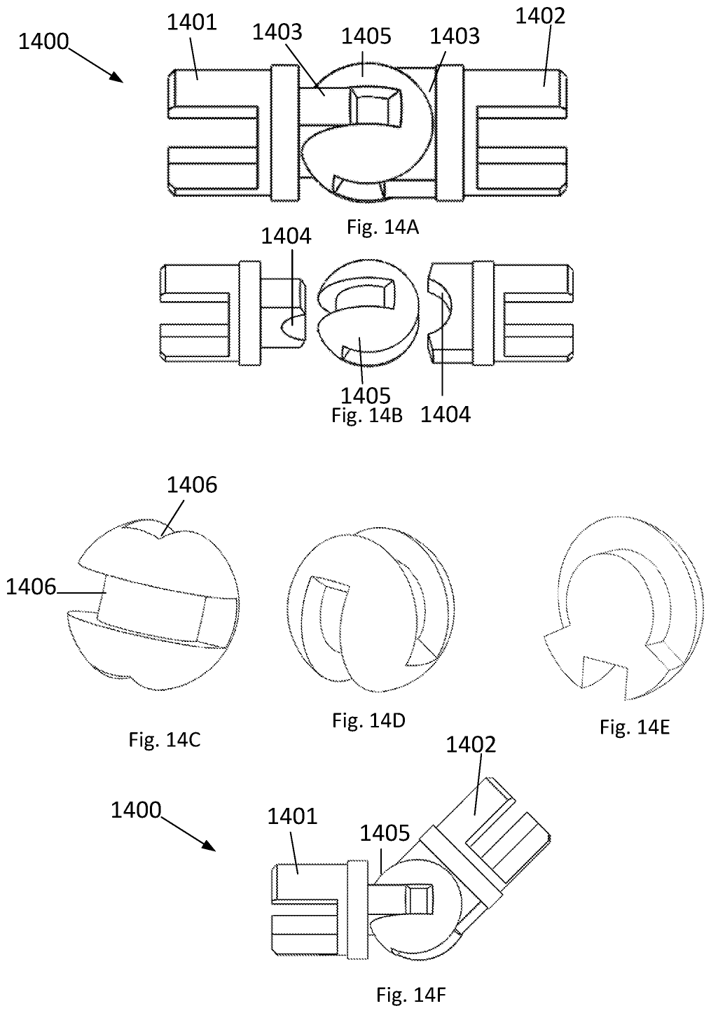

In embodiments, for example as shown in FIG. 11, an articulating sheath 1100 may comprise a single axis articulating sheath comprising a distal portion 1101 including two axle projections 1102, and a proximal portion 1103 including two projections 1104 with bores coupled to the axles in order to form 1 degree of freedom hinged joint. The distal and proximal portions further each include a central through hole defining portions of the lumen of the articulating sheath. This joint is advantageous in that it only includes two parts and thus reduces manufacturing costs compared to joints with three or more parts.

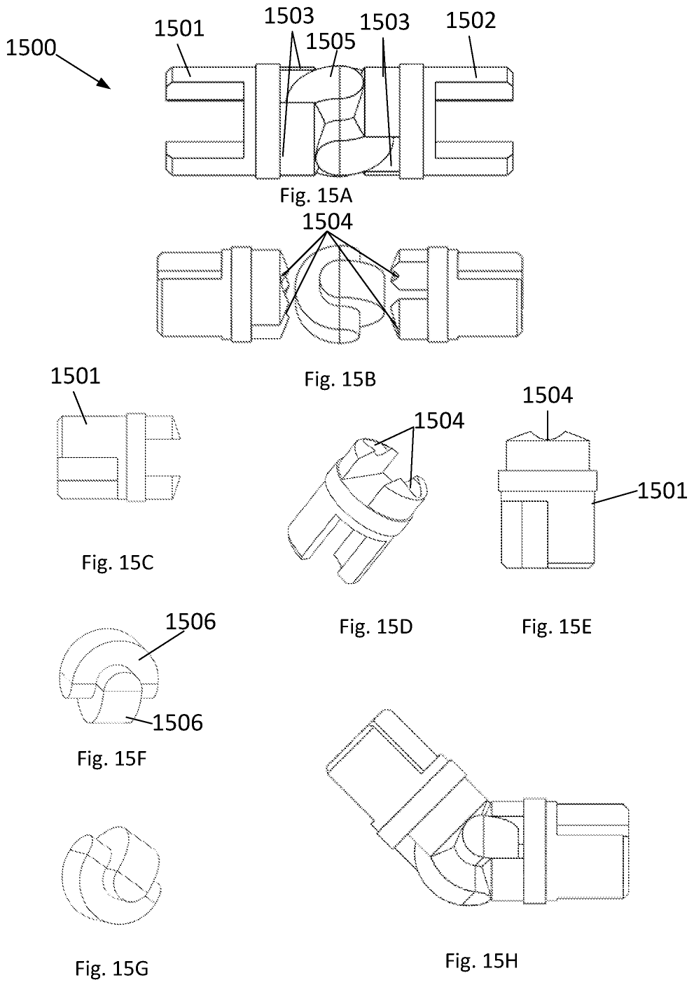

In embodiments, articulating joints may have more than 1 degree of freedom, for example a 2 degree of freedom joint as shown in FIGS. 12A-12F. The 2 degree of freedom joint 1200 comprises a distal portion 1201, a proximal portion 1202, and a central portion 1203. As shown in the exploded view of FIG. 12B, the central portion 1203 comprises four axles 1204 defining two perpendicular axes around a central through hole 1205. The central portion 1203, in addition to the distal portion 1201 and proximal portion 1202 may include radial through holes for tendons and rods, as discussed above. As shown in FIG. 12C, the distal and proximal portions 1201 and 1202 may each comprise two grooved projections 1206 on either side of a central through hole 1205. The grooved projections 1206 of each of the distal portion and the proximal portion are rotationally coupled to the axles 1204 of the central portion, so the proximal portion has 1 degree of rotational freedom with the central portion and the distal portion has 1 degree of freedom with the central portion. The respective 1 degree of rotational freedoms may be perpendicular so that the distal portion and proximal portion have 2 degrees of freedom relative to each other.

As shown in FIGS. 12D-12F each degree of freedom of the 2 degrees of rotational freedom is independent. For example, the distal portion 1201 may be oriented at 0 degrees relative to the central portion 1203, and the proximal portion 1202 may be oriented at 0 degrees relative to the central portion 1203, as shown in FIG. 12D. Further, the distal portion 1201 may be oriented at 0 degrees relative to the central portion 1203, with the proximal portion 1202 oriented at 45 degrees relative to the central portion 1203, as shown in FIG. 12E. Additionally, the distal portion 1201 may be oriented at 45 degrees relative to the central portion 1203, with the proximal portion 1202 also oriented at 45 degrees relative to the central portion 1204, as shown in FIG. 12F.

As shown in FIGS. 12C, the distal and proximal portions 1201 and 1202 each include a central through hole 1205 between the grooved projection 1206. When assembled with the central portion 1203, the central through holes 1205 of each of the three portions define a central lumen of the articulating sheath. The central portion 1203 defining two axes provides a short length for a joint having 2 degrees of freedom, due to the axes intersecting, which allows for a tight degree of articulation. As a result, even in a confined anatomical region, surgeons can use the bone work tool with the 2 degree of freedom joint to offer sufficient articulation with less chance of bumping into the surrounding tissues.

### Articulating Wrist Joint of Torque Transmission Shaft

In embodiments, the portion of the torque transmission shaft extending through the central lumen of an articulating sheath, for example as shown in FIGS. 9A-9H, 10A-10J, 11, and 12A-12F, comprises a wrist joint with 2 or more degrees of freedom in order to allow for articulation of the articulating portion of the shaft while simultaneously allowing the torque transmission shaft to be rotated within the sheath. The wrist joint may be coupled on either end to rigid portions of the torque transmission shaft, and/or to other wrist joints of adjacent articulating portions.

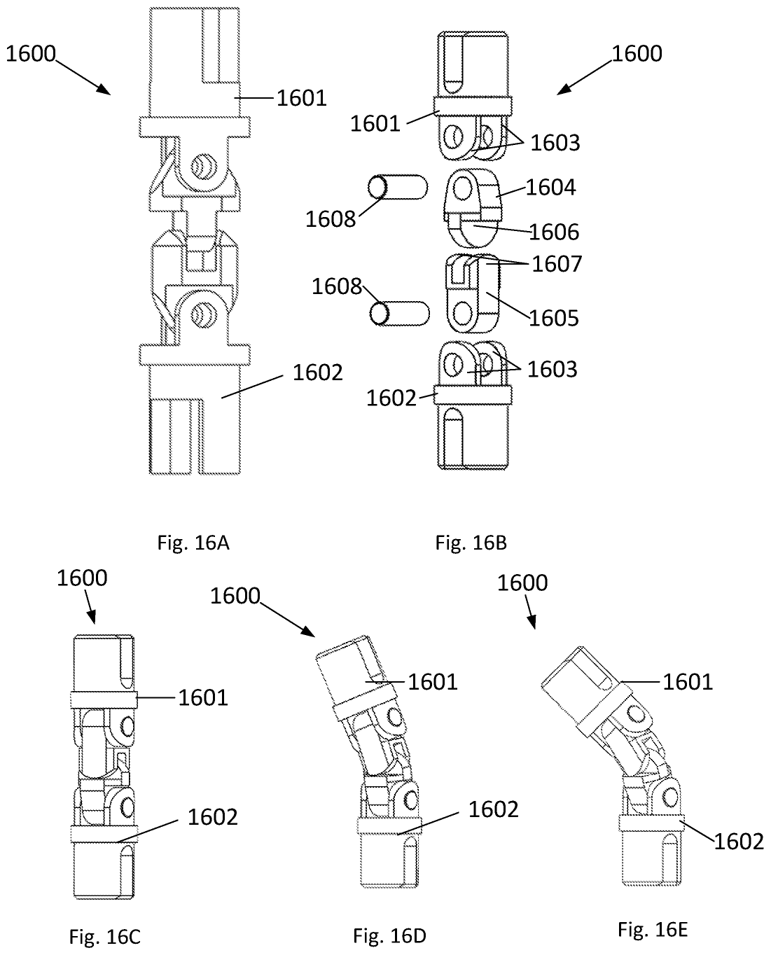

The articulating wrist joint may comprise a universal joint 1300, for example as shown in FIGS. 13A-13C. The universal joint 1300 comprises a distal shaft portion 1301 and a proximal shaft portion 1302 each with projections defining axle bores 1305. The universal joint 1300 further comprises a cross-shaft 1303 comprising four radially positioned axle shafts defining two perpendicular axes. The universal joint 1300 is able to be rotated with the axes of the proximal and distal shaft portions, and axles coupled thereto, being oriented at non 0 degree angles, for example as shown in FIG. 13C, in order to have two degrees of freedom in order to be rotated within the lumen of an articulating sleeve as disclosed above.

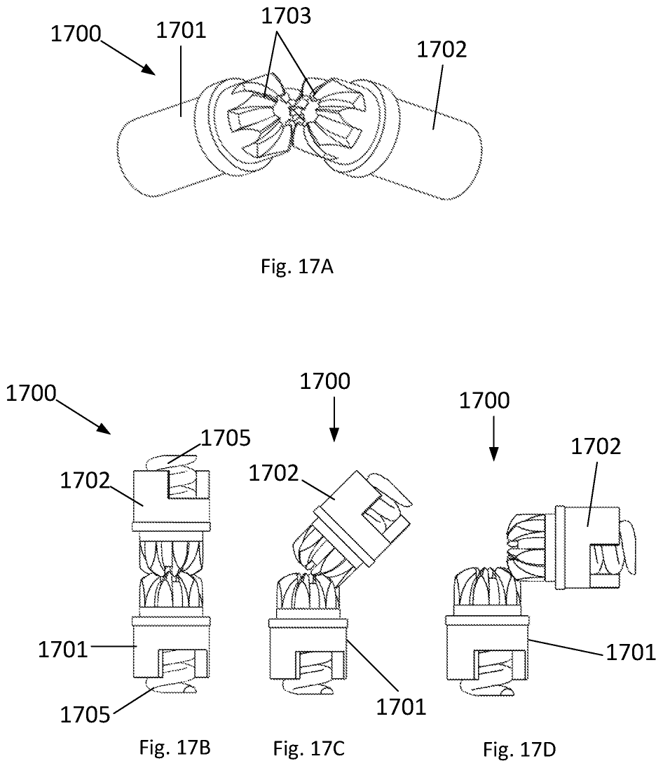

The articulating wrist joint may comprise a slotted ball joint, for example the slotted ball joint 1400 as shown in FIGS. 14A-14F. The slotted ball joint 1400 comprises a distal shaft portion 1401 and a proximal shaft portion 1402 each with a projection 1403 defining a concave surface 1404. The slotted ball joint further comprises a slotted ball 1405 defining two grooves 1406. As shown in FIGS. 14C-14E, the paths of the grooves may be on two perpendicular planes. As shown in the cross-section of FIG. 14E, each groove 1406 follows and arced path and may extend around the ball between 180 and 300 degrees. As shown the grooves 1406 may not intersect. The groove may have a rectangular cross-section in a direction perpendicular to the arced path. The projections 1403 of the proximal and distal shaft portions are each received in respective grooves as shown in FIG. 14A, so that the curved surfaces of the projections 1403 contact the bottom curved surfaces of the grooves 1406 to form two sliding 1 degree of freedom joints. The projections 1403 may have a rectangular cross-section complementary to the cross-section of the grooves 1406. The axis of rotation of each joint is defined by the center point of the curvature of the respective groove. As shown, the axes of rotation of each joint intersects in the center of the slotted ball 1405. The slotted ball joint is able to be rotated with the two axes being oriented at non 0 degree angles, for example as shown in FIG. 14F. Slotted ball joints are advantageous in that they do not include pin axles and corresponding axle bores, which may be difficult to manufacture and/or may be weak when joints are produced in a miniature scale.

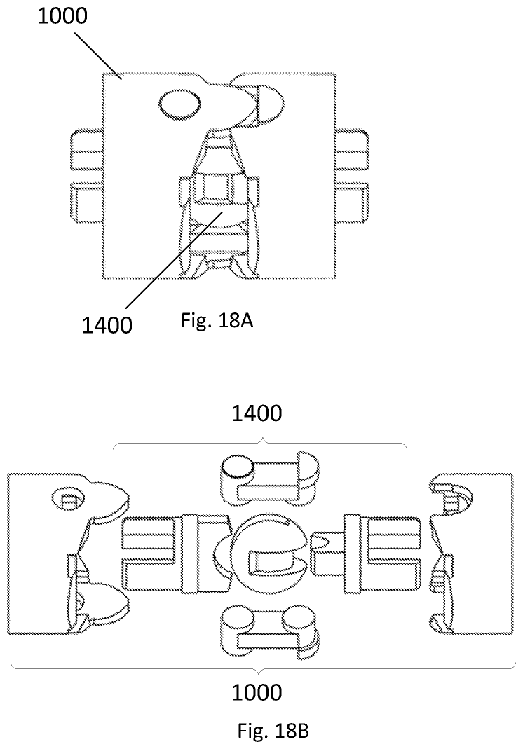

In embodiments, the articulating wrist joint may be a saddle ball joint, for example as shown in FIGS. 15A-15H. As shown in FIG. 15B-15E, the saddle ball joint 1500 comprises a distal shaft portion 1501 and a proximal shaft portion 1502 each with two projections 1503 defining two concave surfaces 1504. The saddle ball joint further comprises a central ball portion 1505 comprising two semi-circular disc portions 1506 coupled perpendicularly. As shown in FIGS. 15A and 15H, the semi-circular disc portions 1506 are respectively received between the two projections 1503 of the proximal and distal shaft portions 1501 and 1502 so that curved end surfaces are received within the concave surfaces 1504 to form two sliding 1 degree of freedom joints. The curved end surfaces each define an axis which intersect each other in the center of the central ball portion. The saddle ball joint is able to be rotated with the two axes being oriented at non 0 degree angles, for example as shown in FIG. 15H. Saddle ball joints are advantageous in that they do not include pin axles and correspond axle bores, which may be difficult to manufacture and/or may be weak when joints are produced in a miniature scale.

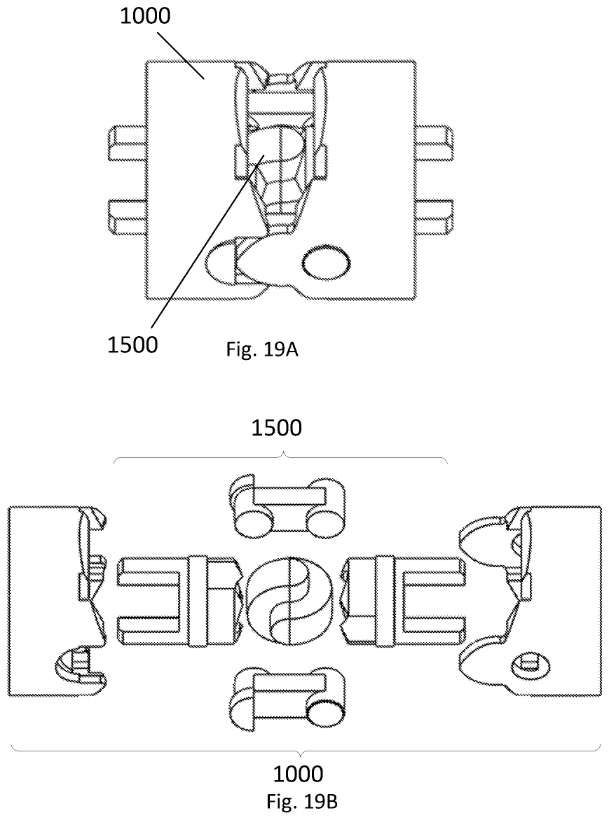

In embodiments, the articulating wrist joint may be a double hinge sliding joint, for example as shown in FIGS. 16A-16E. As shown in FIG. 16A, the double hinge sliding joint 1600 comprises a distal shaft portion 1601 and a proximal shaft portion 1602 each with two projections 1603 each having an axle bore define an axis of rotation. The double hinge sliding joint further comprises a first central portion 1604 and a second central portion 1605. The first central portion 1604 comprises a first end defining an axle bore coupled to the axle bore of the proximal shaft portion with a pin 1608, and a second end defining a central projection 1606. The second central portion comprises a first end defining an axle bore coupled to the axle bore of the distal shaft portion with a pin 1608, and a second end defining two projections 1607 defining a slot. The central projection of the first central portion 1604 is received within the slot defined by the two projections 1607 of the second central portion to form a sliding joint. With the central projection 1606 within the slot, rotation of one of the first and second central portions 1604 and 1605 are transferred to the other through the contacting sliding surfaces of the projections 1606 and 1607.

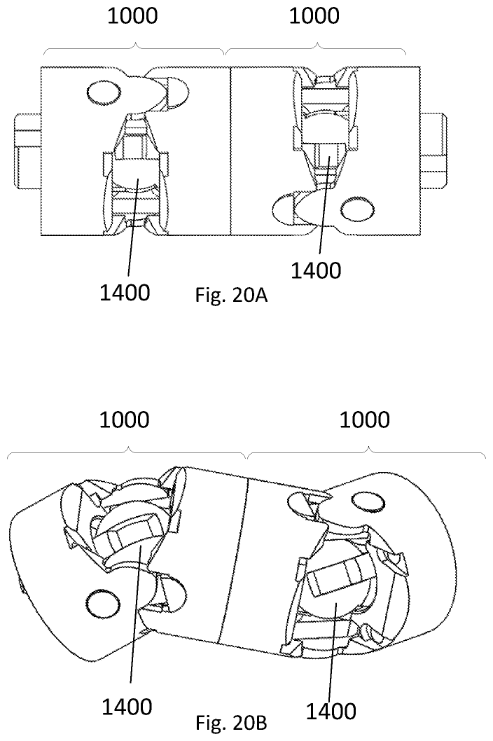

In embodiments, the articulating wrist joint may be a beveled gear joint. As shown in FIGS. 17A-17D, a beveled gear joint 1700 may comprise a distal shaft portion 1701 and a proximal shaft portion 1702 each with a semi-spherical bevel gear 1703. The beveled gears 1703 mesh and allow transfer for rotation when the proximal and distal shaft portions 1701 and 1702 are oriented from 0 degrees to 90 degrees as shown in FIGS. 17B-17D. In embodiments, the teeth of the beveled gears 1703 do not extend all the way to the central shaft axis, as is shown in FIG. 17A. In embodiments, to maintain contact between each pair of semi-spherical bevel gears 1703, a spring 1705 can be used at each spherical gear side to push the two gears against each other.

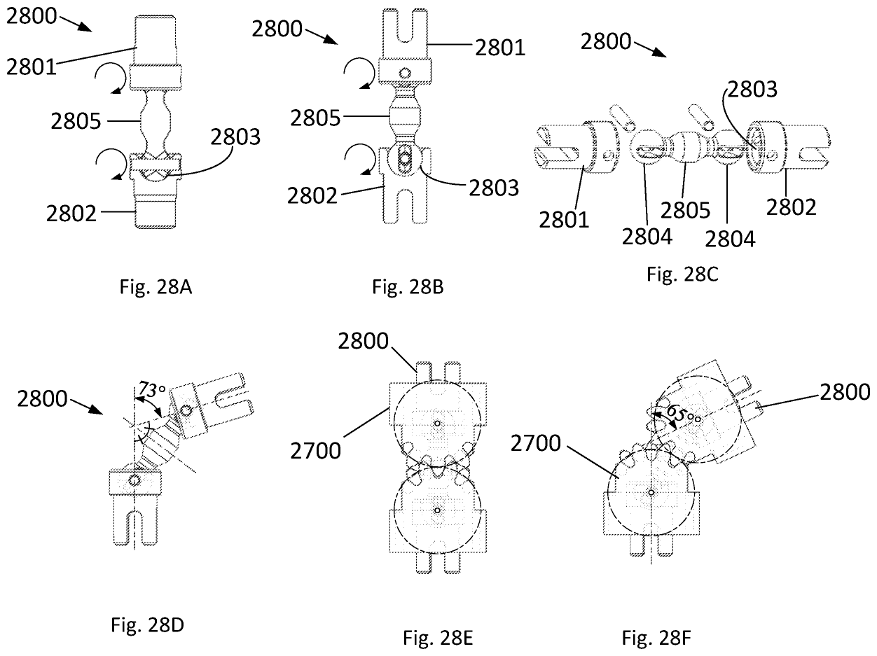

In embodiments, the articulating wrist joint may be a bi-centric wrist joint. As shown in FIGS. 28A-28F, a bi-centric wrist joint may be a double universal joint (U-joint) 2800. As discussed above, wrist joints such as double universal joint (U-joint) 2800 may be used for torque transmission, for example for drilling. As shown, a double universal joint (U-joint) 2800 may comprise a distal shaft portion 2801 and a proximal shaft portion 2802. Each of the distal shaft portion 2801 and the proximal shaft portion 2802 define a socket 2803 for receiving a spherical end 2804 of a middle shaft 2805. Each spherical end 2804 is coupled within a socket 2803 with a pin 2806. The spherical ends 2804 are mounted within the sockets 2803 with the pin 2806 so that the middle shaft 2804 can rotate relative to the distal and proximal shaft portions 2801 and 2802 around a longitudinal axis of the pin 2806, as shown in FIG. 28B. Further, the pins 2806 are coupled within the spherical ends 2804 so that the middle shaft 2805 may rotate around an axis perpendicular to the longitudinal axis of the pin 2806, as shown in FIG. 28A. The combination of these two rotational degrees of freedom defines two rotational degrees of freedom between each of the middle shaft 2805 and the distal shaft portion 2801 and the middle shaft 2805 and the proximal shaft portion 2802. Accordingly, the double universal joint (U-joint) 2800 includes four degrees of rotational freedom between the distal shaft portion 2801 and the proximal shaft portion 2802. Wrist joints with four degrees of rotational freedom are advantageous compared to wrist joints with two degrees of freedom as they allow for greater articulation angles. In embodiments, the double universal joint (U-joint) 2800 may include 73° articulation as shown in FIG. 28D. In embodiments, with the distal shaft portion 2801 and the proximal shaft portion 2802 symmetric about a middle plane, the rotation transmission is constant, namely it is a so-called constant-velocity joint. Constant velocity joints decrease potential vibration caused by the nonlinearity of rotation transmission compared to a single U-joint.

### Sheath and Wrist Combinations

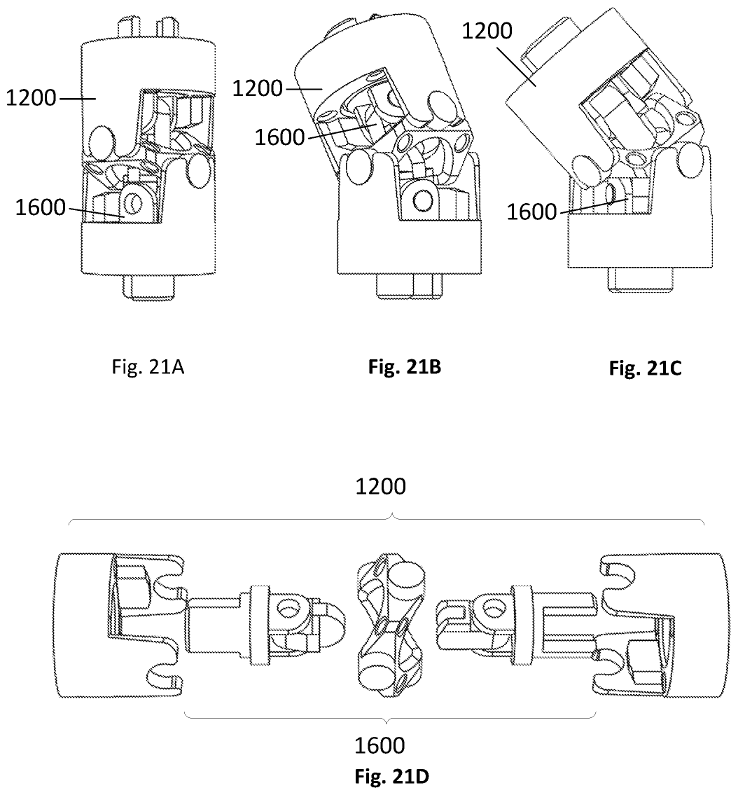

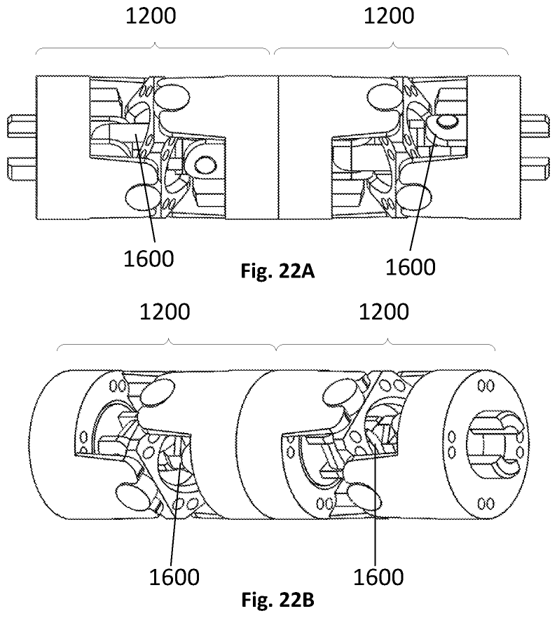

In embodiments, any of the above disclosed articulating sheaths may be used with any of the above disclosed wrist joints. For example, as shown in FIGS. 18A and 18B, the articulating sheath 1000 of FIG. 10A may be used with the wrist joint 1400 of FIG. 14A. Further for example, as shown in FIGS. 19A and 19B, the articulating sheath 1000 of FIG. 10A may be used with the wrist joint 1500 of FIG. 15A.

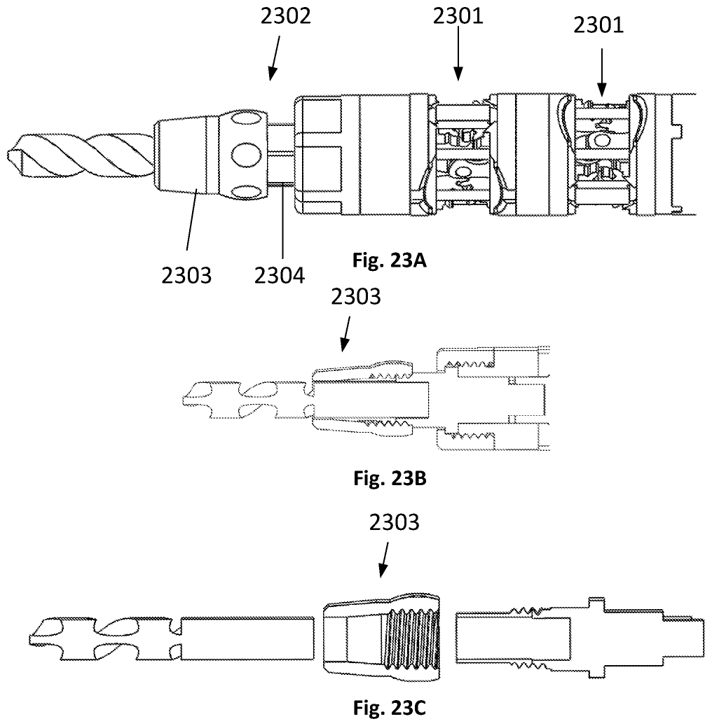

Any combination of articulating portions, including any combination of articulating sheaths and wrist joints, may be directly coupled together immediately adjacent one another. For example, as shown in FIGS. 20A and 20B, two articulation portions each comprising the articulating sheath 1000 of FIG. 10A and wrist joint 1400 of FIG. 14A may be coupled together immediately adjacent one another. As shown in FIG. 20B, each articulating portion may be coupled so that the degrees of freedom are offset, for example perpendicular. As shown, the combination of two articulating portions allows for two perpendicular degrees of rotational freedom with a rotating torque transmission shaft with two 2-DOF articulating portions within the central lumens of the sheathes.

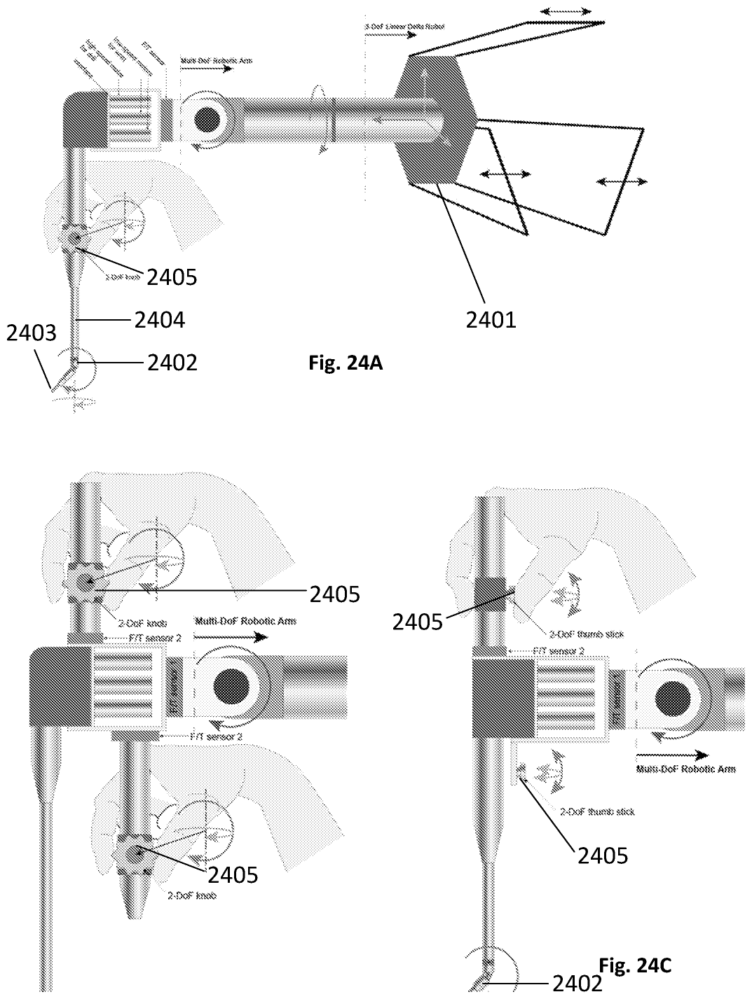

In embodiments, a single wrist joint may be used with sheaths have two or more degrees of freedom. For example, the 2 degree of freedom articulating sheath 1200 of FIG. 12A may be used with the wrist joint 1600 of FIG. 16, as shown in FIGS. 21A-21D. The sliding interface of the central projection within the slot allows for the wrist 1600 to transfer torque when the articulating sheath 1200 if oriented with both axes of rotation being at non 0 degree angles.

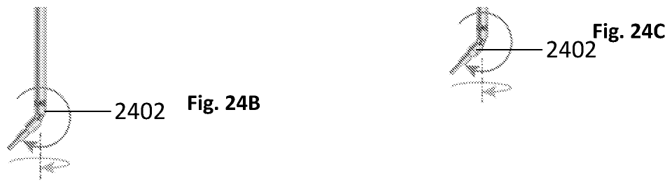

As shown in FIGS. 22A and 22B, two 2-degree of freedom articulating portions, for example as shown in FIG. 21A including two articulating sheathes 1200 and two wrist joints 1600, may be directly coupled together to achieve a 4 degree of freedom joint allow more intricate maneuvering around anatomical obstacles. In embodiments, any number of articulating joints may be directly coupled together, or indirectly coupled together with intermediary rigid sheath and shaft portions between the articulating joints, between the base portion and end effector in order to allow the shaft to conform and navigate around obstacles. Sequential articulating joints may be coupled to each other with any orientation of the respective axes of rotation of the degrees of freedom. For example, two 1 degree of freedom articulating joints may be coupled with the axes at 0 degrees, 90 degrees, 45 degrees, etc.

FIGS. 28E and 28F shows the double universal joint (U-joint) 2800 with the central lumen of the sheath 2700. As shown, the articulating portion formed by this combination of wrist and sheath may allow for articulation of 65° while the shaft is rotating within the central lumen.

### End Effector

The end effector coupled to the distal end of the shaft may be configured to perform various surgical tasks. In embodiments, the end effector may be rotated relative to the sheath of the shaft by the internal torque transmission shaft and be used for boring holes, burring, debriding tissues, and driving screws.

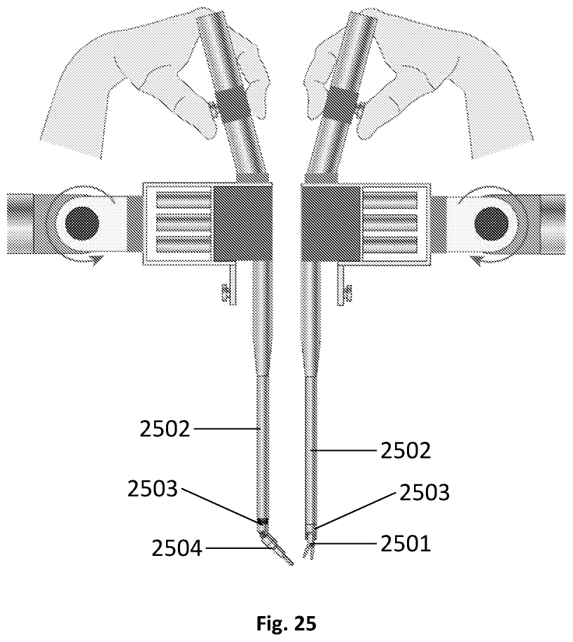

FIG. 23A shows a distal end of a surgical device comprising two articulating joints 2301 and an end effector 2302. The end effector 2302 may comprise a drill bit collet 2303 coupled to the torque transmission shaft 2304 in order to rotate with the torque transmission shaft. The drill bit collet 2303 defines a central bore, a threaded outer surface and two notches along the threaded other surface. The end effector further comprises a threaded collet nut with a conic inner lumen threadably coupled to the drill bit collet. Tightening the collet nut on the drill bit collet causes the central bore to tighten against an inserted drill bit due the conic lumen and notches. The drill bit collet may be rotatably coupled to the sheath with a fixing nut threadably coupled to a threaded distal end of the sheath as shown in FIG. 23B.

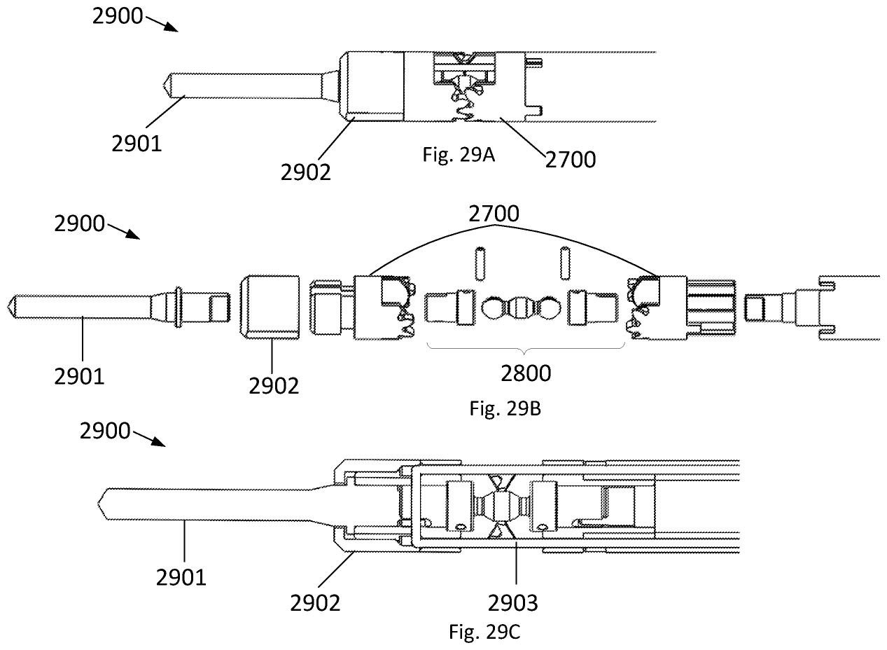

FIGS. 29A-29C show an articulating drill tip assembly 2900 including sheath 2700 and wrist joint 2800, as discussed above. The drilling torque is transmitted from a backend motor to the drill bit through a spinning shaft and the double U-joint 2800. The articulating drill tip assembly 2900 may include an interchangeable drill bit 2901 secured by a nut 2902. The articulating drill tip assembly 2900 may also include a tendon 2903, as discussed above, used to actuate the joint to cause articulation at a desired angle and to ensure the engagements between the distal portion 2701 and proximal portion 2702.

### Handheld Surgical Device

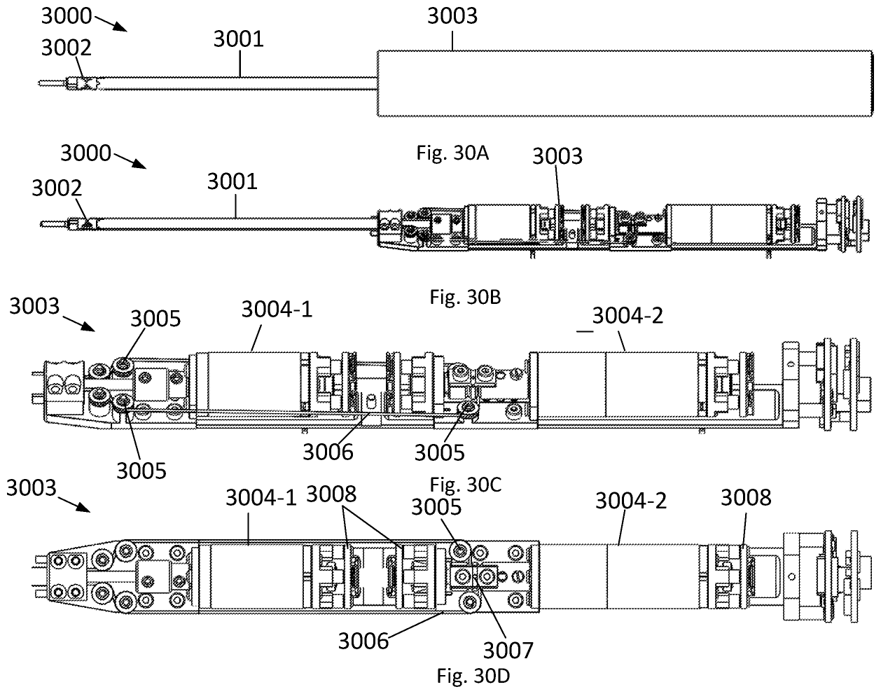

In embodiments, the sheathes, wrist joints, and end effectors, as disclosed herein may be included in a handheld surgical device, for example as shown in FIGS. 6 and 7. FIGS. 30A-30C show an embodiment of a handheld surgical device 3000. The handheld surgical device 3000 includes a shaft 3001 with an articulating portion 3002, as discussed above, and a backend actuation unit 3003. The backend actuation unit 3003 includes two motors 3004. In embodiments, the motors 3004 may have different speeds. For example, the motors 3004 may include a high-speed motor 3004-1 for spinning a drill shaft and a low-speed gearhead motor 3004-2 for driving tendons for the articulating portions of the shaft. A show, pulleys 3005 may be used to guide the tendons 3006 from the instrument shaft to a capstan 3007 installed on the output shaft of the low-speed motor 3004-2 in order to avoid interference with the high-speed motor 3004-1 in the front. A tendon clamp may terminate the tendons with friction. Magnetic encoders 3008 may be included behind the high-speed motor 3004-1 to measure the motor angle for velocity control while two magnetic encoders may be included in front of and behind the low-speed motor 3004-2 to measure the angles of the input and output motor shafts, respectively.

### Robotic System

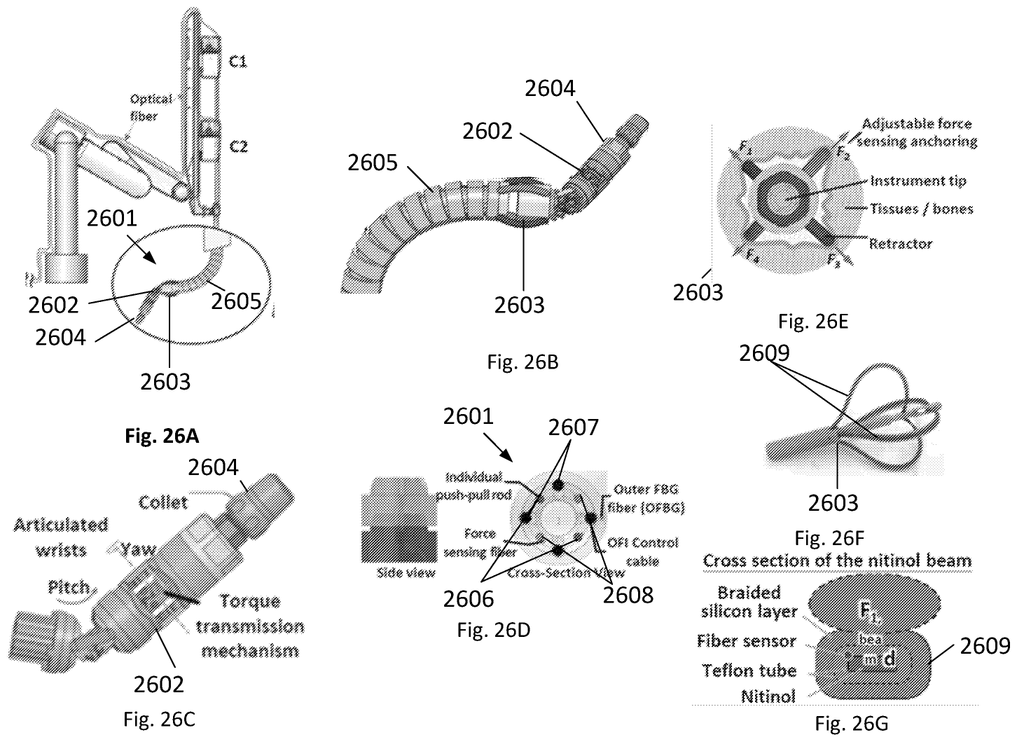

In embodiments, the surgical device may be part of a surgical robotic system. For example, the shaft with an articulating portion and end effector may be integrated into a surgical robotic system as a removable instrument, for example as shown in FIGS. 24A-24C. The base portion 2401 of the surgical portion with the drivers for controlling the articulating joints 2402 and rotating the end effector 2403 may be backend connected to a robotic system. In embodiments, the surgical robotic system may operate in a teleoperation mode, similar to a da Vinci Surgical Robot, or operated in a collaborative control mode, like a Galen Robot.

In embodiments, various user control systems may be used to control the degrees of freedom of the articulating portions. In embodiments, the shaft 2404, including rigid portions and articulating joints 2402, and end effector 2403 may be coupled to a multi-DoF robotic arm as shown in FIGS. 24A-24C. The robotic arm can be a general robotic manipulator, which can be serial or parallel, or even combined. The system may include one or two force/torque sensors (F/T sensors) so that the robot and the articulating joints 2402 of the shaft can be operated in a collaboratively controlled manner. The user controls 2405 may include a 2-DoF knob or thumb stick that located anywhere on the robot, for controlling a 2-DoF articulating portion. For example, 2-DoF knobs may be positioned on the robotic arm proximate to the drill shaft, as shown in FIG. 24B. In embodiments, one DoF of the knob controls the rotation around the instrument shaft, and the other DOF of the knob controls the rotation perpendicular to the instrument shaft.

The 2-DoF knobs of a device may be motorized for providing force feedback and other functions or may be non-motorized. The control system translating the user input to articulating joint output may have damping to make motion of the articulating joints and end effector smoother. In embodiments, and the shaft and user controls may be a module detachable from the robotic arm. The detachable instrument may include electrical contacts connected to the robotic arm providing power and control signals for drivers of the torque transmission shaft. In embodiments, drivers (e.g. motors, linear actuators, etc.) may be provided in the robotic arm which mechanically couple to torque transmission shafts and/or rods in the detachable instrument in order to actuate the articulating portion.

The force measured by F/T sensors of the device may be a combination of the forces from the environment, such as tissues, user hands, and the dynamic forces of all the components located on the left side of the sensor, including the instrument and the motors. Because the robot works in low speed and acceleration the inertia forces, centrifugal and Coriolis forces are minimal. Thus, those forces can be either ignored by a control system due to their minimal effect or compensated by a control system based on an accurate dynamic model of the system, and the position, velocity, and acceleration information from the encoders. Moreover, an inertia measurement unit (IMU) is fixed on the distal end of the robotic arm, which can measure rotational velocity and translational acceleration directly. The gravitational forces can be compensated using the F/T sensor measurement using a mechanical model and only leave the combined force from the environment and user hands. This force is then used as the control input of an admittance controller for the robotic arm.

In embodiments, a 2-DoF knob can be located either upside or downside the arm for the ease of use, instead of on the instrument. In embodiments, the user controls, for example 2-DoF knobs, may be fixed on the robotic arm and located outside of the portion of the instrument that is a sterile interface. During use, the knobs may be draped therefore will not need to be sterilized or be disposed after a procedure. This design approach reduces the cost of the robot as the knob interface can be reused in subsequent clinical uses. In embodiments, a second F/T sensor may be positioned between the handle and the robotic arm. With two F/T sensors, forces may be decoupled from the environment and the user hand. This force signal from the user hand may be used as the control input of an admittance controller for the robotic arm. The force from the environment can be used as an input of another admittance controller for the robotic arm. The parameters of the two controllers, such as damping, inertia, and spring constant, are independent of each other and thus can be adjusted separately to achieve different control behaviors. An advantage of a two-sensor configuration is for a delicate operation on a tissue the spring constant of the admittance controller can be tuned for the environment, in which case the user feels a large force feedback even if a small force is applied on the tissue.

In embodiments, the user input can be 2-DoF joysticks. 2-DoF joysticks can be positioned anywhere a 2-DoF knob can be positioned and can also be integrated with a second force sensor. The difference between a 2-DoF joystick and a 2-DoF knob in controlling the articulating joint is that this joystick cab use a relative position control mode or a velocity control mode. For example, when the joystick is pushed in a direction the input may be used to control the velocity or the relative position of movement of an articulating portion of the shaft, wherein the movement is proportional or follows a self-defined mapping corresponding to the movement of the joystick.

In embodiments, the control system may be used to control non-rotating end effectors 2501 connected to a shaft 2502 with articulating portions 2503, as discussed above. For example the end effector may be forceps, endoscopes, or knives. In embodiments, multiple collaboratively controlled robotic arms can work together at the same time including arms with non-rotating end effectors 2501 and rotating end effectors 2504, as shown in FIG. 25.

### Anchor System

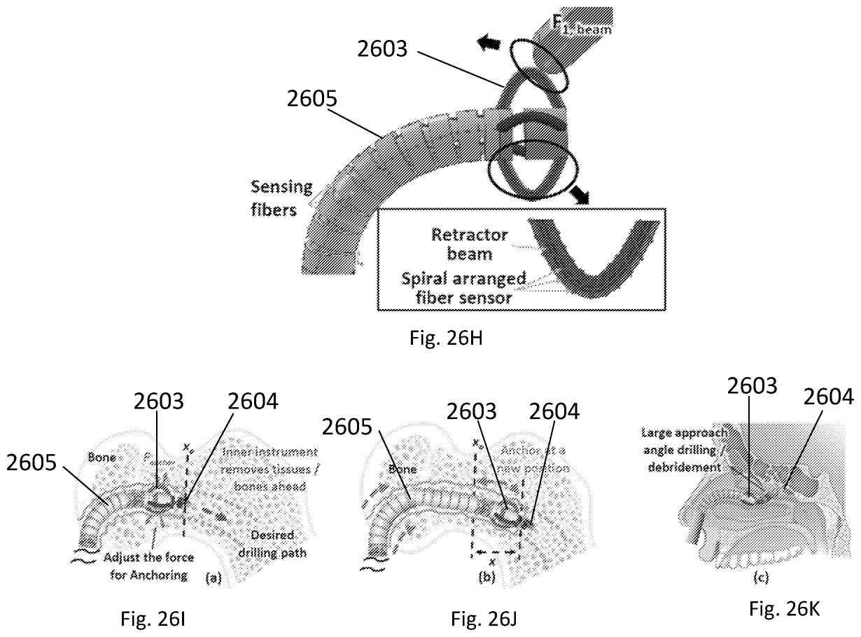

In embodiments, a shaft 2601 including an articulating portion 2602 may be coupled to an anchoring system 2603 so that the articulating portions 2602 are between the end effector 2604 and the anchoring system 2603, for example as shown in FIG. 26A. The anchoring system 2603 may be used to anchor a portion of the shaft within a patient, and may include force sensors to determine the anchoring force. Nested manipulator drilling system with an adjustable anchoring system 2603 integrated with force sensing may be used for curved drilling and higher drilling stability. A shown, a nested manipulator drilling system may comprise a robotic manipulator, two motor packs for driving tendons and rods, and a nested hybrid flexible and articulated drilling manipulator at the distal end. In embodiments, the distal end including portions of the shaft and the end effector may be connected to a passive flexible catheter 2605 inside the outer flexible instrument. Portions of a passive flexible catheter 2605 may extend through a hollow center of the anchoring system 2603. The inner flexible catheter can adjust its shape passively according to the shape of the outer flexible manipulator. The outer flexible manipulator may be driven by tendons or push-pull rods. As shown in the cross-sectional view of FIG. 26D, push-pull rods 2606, tendons 2607, and optical fibers 2608 for shape sensing of the outer flexible manipulator and for force sensing of the adjustable anchoring system pass through the holes at each segment of the outer flexible manipulator.

The adjustable anchoring system comprises a plurality of superelastic nitinol beams 2609 around the end of the outer flexible instrument. The beams may be caused to flex and bow using tendons or push-pull rods in the flexible shaft. The anchoring system can be used to maintain the distal end of a device at a location within a body by pressing the beams against tissue. This anchoring allows precise and stable drilling on hard bones using the end effector distal to the anchoring system. The superelastic beams for the anchoring system may be covered by Teflon tubes and silicon braided layer. Optical fibers may be embedded in the beam covering and used by a controller to sense the interaction force applied on human tissues by the bowed beams when used as anchors.

FIGS. 26H-26K show examples of working procedures using an anchoring system. As shown, the device is inserted into a bodily cavity and the beams for the anchoring system 2603 are adjusted to enable a reliable physical support of the distal end with end effector 2604 of the flexible manipulator. Next, the portion of the device distal to the anchoring system 2603 including the articulating portions and end effector 2604 are navigated through the desired drilling path using the articulating portion(s) while the torque transmission shaft is rotated to cause drilling within the bodily cavity to form or lengthen a bore. Once the bore has been formed or lengthened, the device may be advanced into the bore, or further into the bore, and the anchoring system 2603 reapplied. As shown in FIGS. 261 and 26J, by iterating those two steps, the end effector 2604, e.g. a drill, can be used for drilling a curved bore. As shown in FIG. 26K, the anchoring system 2603 may be used to support the distal end of the device within a bodily cavity, for example a nasal passage, and be used to achieve a large approaching angle for drilling/debridement with the end effector 2604 in other applications.

The various aspects, embodiments, implementations or features of the described embodiments can be used separately or in any combination. In particular, it should be appreciated that the various elements of concepts from FIG. 1-30D may be combined without departing from the spirit or scope of the invention.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, or gradients thereof, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained.

Preferred embodiments of this invention are described herein, including the best mode known to the inventors for carrying out the invention. The invention is susceptible to various modifications and alternative constructions, and certain shown exemplary embodiments thereof are shown in the drawings and have been described above in detail. Variations of those preferred embodiments, within the spirit of the present invention, may become apparent to those of ordinary skill in the art upon reading the foregoing description. The inventors expect skilled artisans to employ such variations as appropriate, and the inventors intend for the invention to be practiced otherwise than as specifically described herein. Accordingly, it should be understood that there is no intention to limit the invention to the specific form or forms disclosed, but on the contrary, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of specific embodiments are presented for purposes of illustration and description. They are not intended to be exhaustive or to limit the described embodiments to the precise forms disclosed. It will be apparent to one of ordinary skill in the art that many modifications and variations are possible in view of the above teachings.

What is claimed is:

1. A surgical device comprising:
a shaft comprising a sheath, an internal torque transmission shaft within the sheath, and an articulating portion comprising an articulating sheath forming a portion of the sheath and an articulating wrist within the articulating sheath and forming a portion of the torque transmission shaft, wherein the articulating portion defines one or more degrees of rotational freedom so that the shaft may rotate around one or more axes at the articulating portion to bend the shaft;
a base unit coupled to a proximal end of the shaft, wherein the base unit is configured to cause the torque transmission shaft to rotate relative to the sheath, and wherein the base unit is configured to actuate the articulating portion to cause the shaft to bend around the one or more axes while the torque transmission shaft is rotating; and
an end effector coupled to the shaft, wherein the end effector is configured to be rotated by rotation of the torque transmission shaft.

2. The surgical device of claim 1, wherein the articulating portion defines exactly one degree of rotational freedom.

3. The surgical device of claim 2, wherein the articulating sheath comprises a bi-centric geared joint comprising a distal portion comprising two geared projections, and a proximal portion comprising two geared projections, wherein the distal portion and the proximal portion are coupled together with dumbbell linkages so that the geared projections of the distal portion mesh with the geared projections of the proximal portion, and
wherein the articulating wrist extends between the geared projections of the distal portion and the geared projections of the proximal portion.

4. The surgical device of claim 2, wherein the articulating sheath comprises a bi-centric non-geared joint comprising a distal portion comprising two teeth, and a proximal portion comprising two notches, wherein the distal portion and the proximal portion are coupled together with dumbbell linkages so that the two teeth of the distal portion mesh with the two notches of the proximal portion, and
wherein the articulating wrist extends between the two teeth of the distal portion and the two notches of the proximal portion.

5. The surgical device of claim 2, wherein the articulating sheath comprises a bi-centric joint comprising a distal portion and a proximal portion each comprising geared portions engaged with each other, and non-geared curved surfaces engaging with each other, and
wherein the articulating wrist defines four degrees of freedom and extends between the distal portion and the proximal portion.

6. The surgical device of claim 3, 4, or 5, wherein the articulating sheath is configured to be rotatable to form a bend in the shaft while allowing for rotation of the articulating wrist within the articulating sheath.

7. The surgical device of claim 2, 3 or 4, wherein the articulating wrist comprises a slotted ball joint comprising:
a distal shaft portion defining a first concave surface;
a proximal shaft portion defining a second concave surface; and
a slotted ball defining a first groove and second groove, wherein the first groove define a first path and the second groove defines a second path, wherein the first path and the second path are on perpendicular planes, and wherein the first concave surface is received within the first groove and the second concave surface is received within the second groove to define two sliding one degree of freedom joints within intersecting axes.

8. The surgical device of claim 7, where the first groove and the second groove each extend between 180 and 300 degrees around a circumference of the slotted ball.

9. The surgical device of claim 2, 3 or 4, wherein the articulating wrist comprises a saddle ball joint comprising:
a distal shaft portion defining a first pair of concave surface;
a proximal shaft portion defining a second pair of concave surface; and
a central ball portion comprising a first semi-circular disc portion and a second semi-circular disc portions coupled together perpendicularly;
wherein the first pair of concave surface are received against the first semi-circular disc portion and the second pair of concave surface are received against the second semi-circular disc portion to define two sliding one degree of freedom joints within intersecting axes.

10. The surgical device of claim 2, 3 or 4, wherein the articulating wrist comprises a spherical beveled gear joint comprising:
a distal shaft portion defining a first spherical beveled gear; and
a proximal shaft portion defining a second spherical beveled gear;
wherein the first spherical beveled gear and the second spherical beveled gear mesh and allow from 0 degrees to 90 degrees of articulation while the beveled gear joint is being rotated.

11. The surgical device of claim 2, 3 or 4, wherein the articulating wrist comprises a universal joint.

12. The surgical device of claim 1, wherein the articulating portion defines exactly two degrees of rotational freedom.

13. The surgical device of claim 12, wherein the articulating sheath comprises:
a distal portion defining a first pair of projections;
a proximal portion defining a second pair of projections; and
a central portion defining a first pair of axles and a second pair of axles around a central opening; wherein the first pair of axles are rotationally coupled to the first pair of projections and the second pair of axles are rotationally coupled to the second pair of projections to define the two degrees of rotational freedom, and wherein the articulating wrist extends between first pair of projections, through the central opening, and between the second pair of projections.

14. The surgical device of claim 13, wherein the articulating wrist comprises a double hinge sliding joint comprising:
a distal shaft portion;
a proximal shaft portion;
a first central portion pivotably coupled to the distal shaft portion and comprising a first projection; and
a second central portion pivotably coupled to the proximal shaft portion and comprising a pair of second projections defining a slot;
wherein the first projection is received within the slot to form a sliding joint configured to allow a transfer of rotation of the torque transmission shaft between the first central portion and the second central portion.

15. The surgical device of claims 1-14, wherein the shaft comprises a second articulating portion comprising a second articulating sheath and a second articulating wrist.

16. The surgical device of claim 15, the second articulating sheath is identical to the articulating sheath, and the second articulating wrist is identical to the articulating wrist.

17. The surgical device of claim 15 or 16, wherein the second articulating portion is directly coupled to the articulating portion.

18. The surgical device of claim 15 or 16, wherein the shaft comprises a rigid sheath portion and a rigid torque transmission shaft portion between the second articulating portion and the articulating portion.

19. The surgical device of claim 15, 16, 17 or 18, wherein an axis of a degree of freedom of the second articulating portion and is perpendicular to an axis of a degree of freedom of the articulating portion.

20. The surgical device of claim 15, 16, 17, 18 or 19, wherein the articulating portion and the second articulating portion bother have exactly two degrees of freedom.

21. The surgical device of any of claims 1-20, further comprising an anchoring system coupled to the shaft, wherein the anchoring system comprises a plurality of beams, wherein the beams are configured to be bowed and pressed against bodily tissue during a procedure in order to maintain a position of a distal portion of the articulating portion so that the end effector can be moved by the articulating portion relative to the anchor.

22. A system comprising:
the surgical device of any of claims 1-21;
a controller; and
a user interface coupled to the controller and configured to allow a user to control actuation of the articulating portion,
wherein the user interface comprises at least one 2 degree of freedom knob, or 2 degree of freedom joystick.

23. The system of claim 22, further comprising:
a robotic arm coupled to the surgical device;
a first force/torque sensor coupled to the robotic arm and the controller; and
a second force/torque sensor coupled to articulating portion of the surgical device and the controller;
wherein the controller is configured to allow the user to control the surgical device and the robotic arm in a collaborative manner based on signals from the first force/torque sensor and the second force/torque sensor.

24. The surgical device of claim 22, where the user interface is configured to be draped while allowing the user to control actuation of the articulating portion during a surgical procedure so that the user interface remains sterile.
